(19) United States
(12) Patent Application Publication
(10) Pub. No.: US 2004/0163655 A1
(43) Pub. Date: Aug. 26, 2004

(54) Method and catheter system applicable to acute renal failure

(75) Inventors: Mark Gelfand, New York, NY (US); Howard R. Levin, Teaneck, NJ (US)

Correspondence Address: NIXON & VANDERHYE, PC, 1100 N GLEBE ROAD, 8TH FLOOR, ARLINGTON, VA, 22201-4714, US

(73) Assignee: PLC Systems Inc., Franklin, MA

(21) Appl. No.: 10/784,807

(22) Filed: Feb. 24, 2004

Related U.S. Application Data
(60) Provisional application No. 60/449,174, filed on Feb. 24, 2003.
(60) Provisional application No. 60/449,263, filed on Feb. 24, 2003.

(51) Int. Cl. A61B 19/00

(52) U.S. Cl. 128/898

(57) ABSTRACT

A method and apparatus for protection of a kidney from damage associated with temporary medullary hypoxia. The treatment is achieved by temporarily and reversibly increasing fluid pressure in the renal pelvis or blood pressure in the renal vein. Increased pressure is maintained at a safe level for the duration of treatment. The steps of the method include: artificially increasing pressure in a urinary tract of at least one kidney of the patient; reducing a renal function of the kidney by maintaining the increased pressure, and reducing the pressure in the urinary tract to increase the renal function above the reduced renal function.

### RELATED APPLICATION

This continuation application claims priority to U.S. Provisional Application Serial No. 60/449,174, filed Feb. 24, 2003, and to U.S. Provisional Application Serial No. 60/449,263, filed Feb. 24, 2002, the entirety of both of these applications is incorporated by reference herein.

### FIELD OF THE INVENTION

This invention relates to a method for preventing and treatment of Acute Renal Failure from such causes as radiocontrast nephropathy or hypotension. It also relates to the reduction of oxygen demand by the kidney by elevating renal vein pressure or renal pelvic pressure. It also relates to the field of pressure-controlled infusion of fluid into a body cavity.

### BACKGROUND OF THE INVENTION

Role of Kidneys in Maintaining Health

The kidneys are a pair of organs that lie in the back of the abdomen on each side of the vertebral column of a mammalian patient, such as a human. The functions of the kidney can be summarized under three broad headings: a) filtering blood and excreting waste products generated by the body's metabolism, b) regulating salt, water, electrolyte and acid-base balance, and c) secreting hormones to maintain vital organ blood flow. Without properly functioning kidneys, a patient will suffer water retention, reduced urine flow, and an accumulation of waste toxins in the blood and body.

The primary functional unit of the kidneys that is involved in urine formation is called the “nephron”. Each kidney consists of about one million nephrons. The nephron is made up of a glomerulus and its tubules, which are can be separated into a number of sections: the proximal tubule, the medullary loop (loop of Henle), and the distal tubule. Each nephron is surrounded by different types of cells that have the ability to secrete several substances and hormones (such as rennin and erythropoietin). Urine is formed as a result of a complex process starting with the filtration of plasma water from blood into the glomerulus. The walls of the glomerulus are freely permeable to water and small molecules but almost impermeable to proteins and large molecules. Thus, in a healthy kidney, the filtrate is virtually free of protein and has no cellular elements. The filtered fluid eventually becomes urine which flows through the tubules. The final chemical composition of the urine is determined by the secretion into and reabsorbtion of substances from the urine required to maintain homeostasis. The two kidneys receive about 20% of cardiac output (total body blood supply) or approximately 800 ml/min of blood. The two kidneys filter about 120 ml of plasma water from blood per minute. This flow rate of filtrate is called the glomerular filtration rate (GFR) and is the gold standard measurement of the kidney function.

Acute Renal Failure

Kidneys are vulnerable to several types of physiologic insults. An insult to the kidney can lead to a serious medical condition called Acute Renal Failure (ARF). ARF is defined as an abrupt reduction of renal function. Irrespective of the cause, impairment of renal function is uniformly associated with high mortality, high cost and few effective treatment options. ARF results primarily from hypotension (low blood pressure). It is also commonly associated with congestive heart failure, sepsis, toxic drugs, complications from surgery and exacerbations of pre-existing renal disease. In all of these conditions, reduced renal oxygen supply eventually leads to ischemia (imbalance of oxygen supply and demand) and cell death in the kidney. This initial ischemic insult triggers production of oxygen free radicals and enzymes that continue to cause cell injury even after restoration of normal blood flow. Tubular cellular damage results in disruption of tight junctions between cells, allowing back leak of glomerular filtrate and further depressing renal function. In addition, dying cells slough off into the tubules, forming obstructing casts, which further decrease GFR and lead to oliguria (low urine production).

Once normal renal blood flow is restored, the remaining functional nephrons increase their individual filtration rate to compensate for the lost nephrons. There are approximately one million nephrons in each normal kidney. Recovery of renal function is dependent upon the size of the remnant nephron pool. If the number of remaining nephrons is below some critical value, continued hyperfiltration results in a vicious cycle with continued nephron loss causing more hyperfiltration until complete renal failure results. This mechanism explains why progressive renal failure is frequently observed even after apparent recovery from ARF. A key to the treatment of ARF and prevention of its progression to chronic renal failure is the reduction of renal ischemia following the initial insult and prevention and reduction of loss of kidney cells (nephrons).

Current Treatment Options and Outcomes

There were more than 360,000 hospitalizations for ARF reported in 1997. This number reflected a 12% increase over the previous year. Approximately 245,000 hospitalized patients had renal failure as a secondary diagnosis. Hospital-acquired ARF occurs in as many as 4% of hospital admissions and 20% of critical care admissions. Most ARF patients are cared for in teaching hospitals (61%). This increased incidence of hospital-acquired ARF is multifactorial. It is related to an aging population with increased risks of ARF, the high prevalence of nephrotoxic exposures possible in a hospital setting, and increasing severity of illness. There are no existing methods of treating ARF once it occurs. Recovery of renal function is dependent on reversal of the original precipitating event(s) and the restoration of renal blood flow. All current treatments of ARF are “supportive” and potentially deleterious. They include: a) support of heart function and blood pressure with drugs, and b) replacement of the fluid and waste removal functions of the kidney with dialysis. Treatments (a and b) can actually increase the severity of the ARF episode (drug-induced renal vasoconstriction, dialysis-induced hypotensive episodes).

ARF is a severe and hard to treat disease. Currently, the mortality rate for hospital-acquired ARF varies from 25-90% depending on the type of ARF and comorbidities of the patient. The mortality rate is 40-50% in general and 70-80% in intensive care settings. Most deaths are not due to the ARF itself but rather to the underlying disease or complications. Mortality rates for ARF have changed little since the advent of dialysis. Interestingly, patients who are older than 80 years with ARF have mortality rates similar to younger adult patients. Pediatric patients with ARF represent a different set of etiologies and have mortality rates averaging 25%.

ARF is not merely a marker of illness. In a follow-up study of 16,000 patients who underwent computed tomography with radiocontrast dye, the mortality rate among those with ARF was 34%, compared with only 7% in a matched cohort from the similarly exposed group. In a recent study, a 31% mortality rate was noted in patients with ARF not requiring dialysis, compared with a mortality rate of only 8% in matched patients without ARF. Even after adjusting for comorbidity, the odds ratio for dying of ARF was 4.9 compared to patients without ARF. Of those who survive ARF, about 50% recover renal function completely, and another 40% have an incomplete recovery. About 5% to 10% develop end-stage renal disease and require maintenance hemodialysis for the rest of their life. In addition to high mortality ARF is associated with high costs to the health system. Only 30% of patients with Acute Renal Failure as a secondary diagnosis were discharged in less than 7 days. Almost 10% of patients with ARF had hospitalizations greater than 30 days. The mean cost per patient episode of ARF is $18,000.

Clearly, there is a large unmet clinical need for a method to prevent and treat ARF regardless of its initial cause or duration. Ideally, a new treatment method would be instituted early enough to prevent loss of any nephrons. Since the duration of ARF is directly related to a continued loss of nephrons, therapies instituted even after the start of an ARF episode may be beneficial in limiting nephron loss before reaching the critical level at which progression to chronic renal failure is assured. Finally, the severity of the disease, both in terms of mortality and morbidity and cost to the health care system, justifies the development and implementation of more aggressive therapies. Specifically, the ideal goals of a new ARF therapy would be to: prevent impending ARF renal failure regardless of etiology; minimize the damage from existing ARF; reduce ICU and hospital days; and reduce mortality and morbidity, and reduce costs.

Types of Renal Insults that Cause Medullary Hypoxia

An episode of hospital-acquired ARF can start with several types of an initial insult and follow different evolutionary scenarios. In all cases the common pathway of damage to the kidney is the ischemia of the kidney medulla. Ischemia is the condition when the oxygen demand by the kidney exceeds the available oxygen supply.

The role of ischemia, also called hypoxia (oxygen starvation), in the progression of ARF is described by Mayer Brezis in the “Hypoxia of the renal medulla—its implications for disease.” (New England Journal of Medicine, Vol. 322, No 10, Mar. 9, 1995 pp. 647-654). Brezis emphasized that in a land animals, a major task of the kidney is to reabsorb water to allow survival in dry environment. Water conservation is implemented by renal medulla where the blood plasma filtrate is concentrated into urine. In the kidney, filtration of blood occurs through a relatively porous membrane and is driven by the hydrostatic pressure of aortic blood. In contrast, the reabsorbtion of water and salt occurs across a very tight membrane that rejects small molecules. As water is reabsorbed, urine is concentrated in the tubules of the kidney. Concentration of small soluble molecules (solute) in urine can be tens of times higher than in the blood plasma. As a result, the kidney spends large amount of energy to reabsorb water and ions. Water reabsorbtion is opposed by the osmotic gradient also called osmotic pressure. This unique gradient of osmolality is largely responsible for the high demand for oxygen (source of energy) by the kidney. Scientific literature shows that the oxygen demand by the kidney is directly and linearly proportional to the GFR. This is easy to explain since almost all (more than 95%) of the GFR is reabsorbed back into the blood.

The body has mechanisms of controlling GFR independently from the renal blood flow of the kidney. Under normal conditions kidney will attempt to maintain GFR constant based on the physiologic need to concentrate urine. At the same time certain physiologic conditions cause the reduction of GRF independently of the blood flow through the kidney. If the ratio of blood flow (determinant of oxygen supply) to the GFR (determinant of oxygen demand) is increased, the hypoxia of the kidney and the resulting ARF can be averted.

There is a long felt need to protect the kidney from various types of acute insults that cause renal damage predominantly by the mechanism of medullary hypoxia and to protect the kidney by reducing the oxygen demand. Further, the oxygen demand can be reduced independently of the perfusion pressure and blood flow of the kidney by reducing a) the rate of reabsorbtion of the filtrate and b) the concentration of solute in the urine collected in the kidney. The rate of reabsorbtion is practically equal to the GFR and therefore any physiologic mechanism to reduce the GFR will also reduce the oxygen demand of the kidney. The following three types of acute renal insults were identified as acute and ischemic in their nature. All three commonly result in ARF.

1. Hypotension During Vascular and Cardiac Surgery

Surgical procedures such as aortic aneurysm repair, surgery on the heart and surgery involving renal arteries often result in the interruption or reduction of blood flow to the kidneys. Patients, especially elderly, ones with chronic renal impairment and diabetes often suffer ARF as a result of such surgery.

2. Radiocontrast Induced Nephropathy

Intravascular iodinated radiocontrast solution (contrast for simplicity) is opaque to x-rays and enables the circulatory system arteries and veins to be visualized. Iodinated contrast is used in such common medical procedures as diagnostic angiography and percutaneous transluminal coronary angioplasty (PTCA). Unfortunately, the use of a contrast agent is associated with a significant incidence of ARF called contrast nephropathy. The exact nature of contrast nephropathy is unknown. Nevertheless, the imbalance between the oxygen supply and demand and the resulting medullary hypoxia plays significant role in contrast nephropathy. The hypoxic nature of contrast nephropathy is described in the “Pathophysiology Of Radiocontrast Nephropathy: A Role For Medullary Hypoxia” by Heyman et. al. (Investigative Radiology, Volume 34(1). November 1999, page 685).

3. Hypotension from Heart Failure and Shock

The resting arterial blood pressure in a healthy human is 120/80 mmHg. When the blood pressure becomes too low, it can result in inadequate perfusion of the heart, brain, kidneys and other vital organs. Low blood pressure, called hypotension, is usually defined as any condition in which the blood pressure is lower than 90/60 mm Hs. If the hypotension is severe or prolonged, and is associated with evidence of vital organ dysfunction, the patient is then said to be in “shock.” In the hospital, severe prolonged hypotension can result in the hypoperfusion (reduced blood flow) of the kidneys and in ARF. In patients in an intensive care situation, such episodes of hypotension can be caused by blood loss (hypovolumea), heart failure and vasodilatation of blood vessels as a result of sepsis of poisoning. Commonly, the medullary hypoxia plays the major role in the progression of ARF.

### SUMMARY OF THE INVENTION

As described above, hospital acquired ARF can be caused by an insult such as an intravenous radiocontrast infusion, an interruption or reduction of blood supply or blood pressure to the kidney during surgery or acute systemic hypotension. In all cases, the common pathway of damage to the kidney is the ischemia of the kidney medulla. Ischemia is the condition when the oxygen demand by the kidney exceeds the available oxygen supply. In a patient with the reasonably functioning heart and lungs, oxygen supply to the kidney is determined by the renal blood flow. Conventional device-based ARF treatment strategies focus on the supply side of the ischemic misbalance. Traditionally, the goal of the ARF treatment in an intensive care unit (ICU) is to increase the supply of oxygenated blood to the kidney by improving renal blood flow and arterial blood pressure. A new method and system have been invention that, contrary to conventional wisdom, treat ARF by reducing the renal metabolic demand for oxygen to prevent or limit cell damage and loss.

To treat ARF resulting from any one of the three insults to the kidney outlined above, several clinically useful and practical embodiments are disclosed herein that allow temporary reduction of the renal oxygen demand. The treatment increases renal blood flow to increase the ratio of oxygen supply to oxygen demand of the kidney by primarily decreasing the demand. The oxygen demand of the kidney may be reduced by at least partially, temporarily and reversibly impeding the ability of the kidney to filter blood (as indicated by measurable GFR and concentrate urine). During the treatment, GFR itself can be temporarily reduced by: increasing renal vein blood pressure, or increasing urine pressure in the pelvis of the kidney. Commonly in animals and humans, these intervention treatments cause significant reduction of the GFR. If prolonged beyond some reasonable time period or allowed to expand beyond some reasonable “physiologic” range, these treatments can cause damage to the kidney. But if tightly controlled and applied for a relatively short time, these interventions can save the kidney from ARF.

Increasing Renal Vein Pressure

In the “Effect of increased renal venous pressure on renal function” (Journal of Trauma: Injury, Infection and Critical Care 1999, December; 47(6): 1000-3) Doty et. al. described effects of elevated pressure in the renal vein on the blood flow and GFR of the kidney. Doty concluded that in the experimental 20 kg pigs, elevation of renal venous pressure (RVP) to 0-30 mm Hg above baseline resulted in the significant decrease in renal artery blood flow (RBF) index from 2.7 to 1.5 mL/min per gram (of kidney mass) and GFR from 26 to 8 mL/min compared with control. Importantly, these changes were partially or completely reversible as RVP returned toward baseline. The GFR of the treated kidney decreased by 70% while the RBF decreased only 45%. The ratio of RBF (index of oxygen delivery) to the GFR (index of oxygen consumption) almost doubled from 0.10 to 0.18.

Similar conclusions can be reached by studying clinical experience with the disease known as an acute abdominal compartment syndrome (AACS). Patients with AACS often have elevated renal vein blood pressure due to partial occlusion or compression of the renal vein. It was observed that in the patients with the renal vein pressure elevated by 30 to 60 mmHg over baseline the kidneys stop making urine but generally are not permanently damaged. Renal function is promptly restored in these patients when the surgeon relieves the abdominal compression. In patients that, as a result of the compartment syndrome, had renal vein pressure elevations of more than 60 mmHg, the kidneys were often damaged temporarily or even permanently.

In normal humans, baseline renal vein pressure is between 0-5 mmHg. Patients with right side heart failure that have chronically elevated venous pressure of 20-30 mmHg often exhibit diminished renal function and reduced renal blood flow. However, even if the exposure to this increased pressure is prolonged over weeks or months, if the renal vein pressure is reduced, the renal function is known to improve as long as the renal vein pressure did not exceed 60 mmHg.

Based on the physiologic response of the kidney to the elevated renal vein blood pressure, commonly perceived as a disease rather than a cure, a counterintuitive method and system has been invented to protect human kidneys from medullary hypoxia. The method comprises temporarily, partially and controllably obstructing venous blood outflow from at least one kidney to prevent or reduce the severity of ARF.

Increasing Urine Pressure in the Pelvis of the Kidney

The renal pelvis (pelvis) is a cavity in the middle of the kidney that is an extension of the ureter. The urine formed in the nephrons of the kidney drains into the renal pelvis. From the pelvis, it drains into the bladder via the ureters. The pelvis, the ureters and the bladder form one cavity. In a normal subject, the pressure in the pelvis of the kidney is at approximately the atmospheric level or slightly above it. During urination the bladder contracts and the bladder pressure can peak as high as 100 cm of water. Unless there is an obstruction in the ureter, the pelvis pressure is elevated significantly for a prolonged time only if the bladder is full.

The physiologic responses of the kidney to the elevated pelvic pressure were investigated in relation to the disease called “obstructive nephropathy”. The term obstructive nephropathy is used to describe the functional and pathologic changes in the kidney that result from obstruction to the flow of urine, raising renal pelvic, and eventually intrarenal pressure to very high levels. Obstruction to the flow of urine can occur anywhere in the urinary tract and has many different causes. Significant obstruction to the flow of urine over a long period of time (a day to weeks) can result in renal failure and need surgical correction. Obstructive nephropathy is responsible for approximately 4% of end-stage renal failure.

At the same time, obstruction of the urine flow and the associated increase of pelvic pressure for a short period of time (hours to a day) seem to be harmless. In the “Reflux and Obstructive Nephropathy” James M. Gloor and Vicente E. Torres reported the recovery of renal function after the relief of complete unilateral ureteral obstruction of variable duration. The recovery of the ipsilateral glomerular filtration rate after relief of a unilateral complete ureteral obstruction has been best studied in dogs and depends on the duration of the obstruction. Complete recovery always occurs after one week of obstruction, although the more prolonged the obstruction, the more prolonged the duration of renal dysfunction prior to total recovery. It takes from days to months of obstruction to induce permanent damage to the kidney. Based on this data, obstruction of urine outflow from one or two kidneys for several hours should have no long-term effect on the kidneys.

The acute effect of elevated renal pelvis pressure on the function of the kidney was studied in animals. Hvistendahl et al described effects of the increased urine pressure on renal function in “Renal hemodynamic response to gradated ureter obstruction in the pig” (Nephron 1996; 74(1): 168-74). Hvistendahl reported that elevation of the ureteral pressure in steps of 10 mm Hg to a maximum of 80 mm Hg decreased ipsilateral Renal Blood flow (RBF) (meaning that blood flow decreased to the kidney in the same side of the body in which the intervention was performed) by 45% from 300 to 168 ml/min. Contralateral (the opposite side of the body or the kidney without intervention) RBF did not change significantly. The mean arterial pressure was constant during the experimental procedures, suggesting that the decrease of RBF was due to a significant increase in ipsilateral renal vascular resistance. Concomitantly with these changes ipsilateral GFR was reduced by 75% from 40 to 10 ml/min.

Notably, in concert with the goal of the invention, the GFR of the treated kidney decreased by 75% while the RBF decreased by 44%. The ratio of RBF (index of oxygen delivery) to the GFR (index of oxygen consumption) increased by 120% from 7.5 to 16.8. Numbers in the Hvistendahl ureter obstruction reference are different from the Doty renal venous blood experiment cited earlier since Doty normalized RBF to the weight of the kidney and Hvistendahl didn't but the end effects of both experiments are strikingly similar: particularly, the ratio of RBF to the GFR approximately doubled as a result of the intervention. In the contralateral kidney, GFR was unchanged during the experiment.

Lelarge et. al. in the “Acute unilateral renal failure and contralateral ureteral obstruction” (American Journal of Kidney Diseases. 20(3): 286-8, 1992 September) reported anecdotal clinical evidence supporting the invention. After obstetrical surgery, a female patient developed an acute failure of one kidney. The ureter of the other kidney was inadvertently ligated (clamped) during surgery. Surprisingly it was the kidney that was not ligated that developed the ARF. It is possible that the ligation of the ureter of the kidney resulted in the increase of the renal pelvic pressure that protected the ligated kidney from an insult from surgery.

Based on these physiologic data points it is reasonable to conclude that the elevation of the renal pelvic pressure to approximately 10 to 80 mm Hg for the duration of the continuing acute renal insult such as hypotension, surgery or contrast infusion will protect the kidney from ARF by increasing the ratio of oxygen supply to oxygen demand in the medulla of the kidney.

Adjuncts to Therapy

If the distension of the bladder or ureter by the elevated pressure becomes painful to the patient, a pain reducing or anti-spasmodic medication can be added to the fluid infused into the bladder, ureters and renal pelvis or given systemically to the patient. To enhance to effect of the drug Electromotive drug administration can be used. Electromotive drug administration (EMDA) involves the active transport of ionized drugs such as the potent local anesthetic lidocaine by the application of an electric current. Rosamilia et. al. described successful painless bladder distension in women using EMDA (“Electromotive drug administration of lidocaine and dexamethasone followed by cystodistension in women with interstitial cystitis”, International Urogynecological Journal Pelvic Floor Dysfunction 1997; 8(3): 142-5). In addition to protecting kidney with pressure, the invention can be supplemented by the irrigation of the renal pelvis with a cold fluid. Cooling of the kidney will reduce the kidney metabolism and further reduce the oxygen demand.

### SUMMARY OF THE DRAWINGS

A preferred embodiment and best mode of the invention is illustrated in the attached drawings that are described as follows:

FIG. 1 illustrates the treatment of a patient by increasing renal vein pressure

FIG. 2 illustrates the treatment by increasing bladder pressure

FIG. 3 illustrates the treatment by increasing the renal pelvic pressure

FIG. 4 illustrates a way to control the bladder pressure

FIG. 5 illustrates an active control of bladder pressure with a closed loop pump system

FIG. 6 illustrates an algorithm for controlling the bladder pressure

FIG. 7 illustrates cooling of the kidney by irrigation of the renal pelvis with cold solution

### DETAILED DESCRIPTION OF THE INVENTION

For the proposed clinical use, the novel method and system can be used to protect a kidney of a patient from an insult that can cause renal ischemia, renal medullary hypoxia, and ARF. These systems and methods can be also used to improve the outcome of the ARF by reducing the damage to the kidney if used before, during or directly following the insult. The insult can be the low arterial blood pressure or the infusion of radiocontrast in blood, such as by using a contrast injector 109. It is also understood that the systems and methods can achieve substantially the same goal of temporarily reducing oxygen consumption of the kidney and GFR while increasing RBF to GFR ratio of at least one kidney. For example, the oxygen demand of the kidney(s) is temporarily substantially decreased to protect the kidney from hypoxia. It is expected that the kidney may not be able to concentrate urine and reabsorb sodium from filtrate back to blood during and directly following the application of the inventive therapy method and system. Although normally an indication of a reduced renal function, these effects are expected to be protective for a kidney that is subjected to a much more serious hazard when applied in a controllable reversible way for a relatively short period of time.

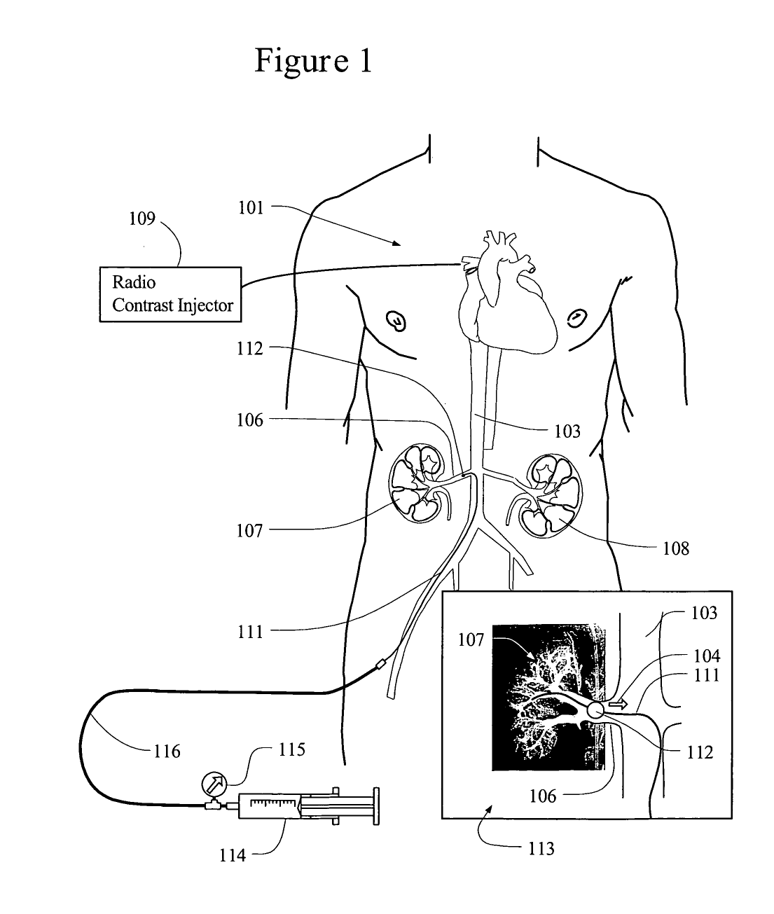

FIG. 1 illustrates one treatment of a patient 101 to protect the kidney 107 from ARF with a system for increasing renal vein pressure. The patient may be selected from a group of patients undergoing contrast injection. The patient may be selected from the group because he is suffering from one or more of a group of illnesses consisting of chronic renal disease, diabetes and old age or other criteria which indicates that the patient is at particularly risk during injection of a contrast agent. This system achieves elevated renal vein pressure by partially occluding the renal vein. The system in its most basic form comprises a vascular catheter 111, an inflatable balloon 112 on the distal (remote, farther from the operator) end of the catheter and the balloon inflation device 114 connected to the proximal (nearest or closer to the operator) end of the catheter.

The system temporarily increases renal vein pressure by creating a removable obstruction of the renal vein. The obstruction is controllable so that it creates the renal artery backup pressure of above normal and for example in the range of 30 to 60 mmHg by partially obstructing but not totally blocking the renal vein outflow. Within the scope of this application words occluding, blocking or obstructing have the same meaning when applied to a body fluid passage.

By way of example, the invention the catheter 111 is inserted into the femoral vein of the patient from an incision or puncture in the groin area. The catheter has outer diameter of up to 9 French but preferably 5 French or less. The catheter is advanced downstream (towards the heart) first into the femoral vein and further into the inferior vena cava (IVC) 103. During the insertion of the catheter, the balloon 112 is deflated and collapsed so as not to interfere with the blood flow and to allow passage through small openings and vessels. Using common fluoroscopic or ultrasonic navigation and interventional tools such as guide wires and guiding catheter sheaths, the distal tip of the catheter 111 is inserted into the renal vein 106 and inflated there.

Panel 113 of the FIG. 1 further illustrates the catheter balloon position in the renal vein 106 of the kidney 107 using a renal venogram (contrast enhanced X-ray image). The renal vein in humans is approximately 8 to 12 mm in diameter at the junction to the IVC. Therefore, when inflated, the balloon 112 should expand to a diameter of approximately 5 to 8 cm to effectively partially occlude the renal vein 106. This partial occlusion creates resistance to blood flow draining from the kidney 107 towards the IVC 103. As a result of this increased resistance, pressure in the renal vein segment between the kidney and the balloon (upstream renal vein pressure) is elevated. Because of the elevated renal vein pressure, the GFR of one or both kidneys 107 is reduced to prevent or reduce the severity of ARF. Pressure below the balloon (downstream renal vein pressure) is approximately equal to the IVC pressure.

The contralateral kidney 108 may not be protected in the embodiment shown in FIG. 1. It is assumed that the contralateral kidney will make urine and have normal GFR during the procedure. If the unprotected kidney is damaged, it is likely to recover on its own over time, while the protected kidney 107 performs normal renal functions. In an alternative embodiment, both kidneys can be protected in the same way using catheters 11.

The proximal end of the catheter 111 is attached to the balloon inflation device 114 by a flexible tube 116. The catheter 111 can include a balloon inflation lumen and pressure conducting lumen for renal vein blood pressure measurement. A syringe 114 balloon inflation device is one example of a device to inflate the balloon 112. Merit Medical Inc. (South Jordan, Utah) offers a wide variety of these type inflation devices for balloon tipped catheters that can be easily adopted for the renal vein obstruction system. For example, Merit Medical manufactures an IntelliSystem Inflation Syringe for balloon catheters used in interventional cardiology to inflate angioplasty balloons inside the coronary arteries of the heart.

The inflation syringe is equipped with the pressure gage 115 to display the renal vein pressure. When inflated, the balloon 112 partially occludes the renal vein thus impeding flow of blood from the kidney veins into IVC 103. The distal end of the catheter 111 can penetrate into one of the smaller veins of the kidney to prevent migration of the balloon into IVC with the venous blood flow 104. It is understood that other ways to anchor the catheter in place can be designed by an experienced catheter engineer. The balloon 112 is positioned near the junction of the renal vein 106 and the IVC 103. It is understood that the balloon can partially or completely reside in the IVC and efficiently impede the outflow of blood from the junction.

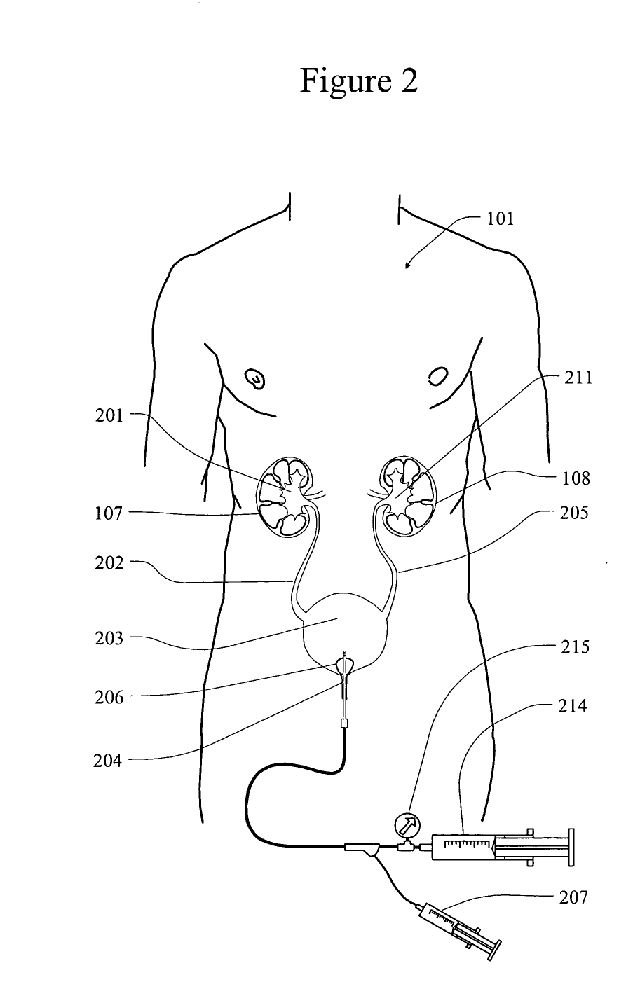

FIG. 2 schematically shows the patient 101 suffering from a renal insult treated with a catheter 204 inserted into the bladder 203. In accordance with another embodiment of this invention, pressure in the patient's bladder 203 is elevated by the controlled infusion of fluid from the catheter. The bladder 203 is connected to the renal pelvis 201 of the first kidney 107 by the ureter 202 and to the renal pelvis 211 of the second kidney 108 by the ureter 205. Together the bladder 203, renal pelvis 201 and the ureter 202 form the urinary tract of the kidney and are in fluid communication.

To increase pressure in the renal pelvis 201, a catheter 204 is placed in the bladder 203. The catheter can be a standard or modified so-called “Foley catheter.” The infusion device 214 is used to infuse fluid such as sterile saline under pressure into the bladder and maintain bladder (and thus ureteral and renal pelvic) pressures at the desired level. The catheter may, for example, increase urinary tract pressure at least to a pressure of 10 to 20 cmH₂O above the urinary tract pressure prior to the artificial increase in pressure. The catheter 204 can be equipped with an occlusion balloon, pressure sensing lumens and drainage lumens in addition to the fluid infusion lumen. A variety of suitable catheters are available from the Bard Medical Division of C. R. Bard Inc. that is a market leader in urological drainage systems. For example a Bardex® Lubricath® 3-Way Catheters can be adopted for the delivery of fluid under pressure into the bladder of a patient and pressure monitoring to ensure safety. The balloon inflation lumen of the catheter 204 can be connected to the external balloon inflation device 207.

It is possible to significantly elevate the pressure in the bladder 203 of the patient by simply occluding the bladder outlet and letting the urine pressure build up on its own. To accelerate the pressure buildup in the bladder, a sterile fluid from the pump 214 may be infused into the bladder via a lumen in the catheter 204. Pressure gage 215 is used to monitor the pressure in the lumen and the bladder. To maintain the bladder pressure at the desired level, for example 60 mmHg, the operator may periodically add or drain some amount of fluid from the bladder.

Similar to the treatment discussed above with respect to renal vein pressure, the bladder pressure (or pressure in one or both of the urethras) is artificially increased to affect the kidney function. Thereafter, a contrast agent may be injected into the blood vessels of the patient. After the contrast agent is dissipated in the patient (or after some other predetermined time period), the bladder or urethra pressure is reduced to its normal level to allow the kidney function to return. These steps may be preformed sequentially, or the bladder pressure may be elevated with or shortly after the contrast is injected.

This method of elevating the pressure in the bladder has the advantage of simplicity. In addition, this method prevents or minimizes AFT in both kidneys as elevated pressure in the bladder affects both kidneys. However, since the flow of urine from the bladder is obstructed, the patient cannot urinate during the treatment. Therefore this embodiment can be only applied for relatively short periods of time (for example up to 24 hours). Alternatively, an “artificial kidney” also called dialysis machine can be used to substitute for the “shut down” kidneys. For example a state of the art device such as the Prisma CRRT machine manufactured by Gambro AB (Stockholm, Sweden) can be used to remove excess fluid and toxins from the patient's body while the patient's kidneys are protected from the insult.

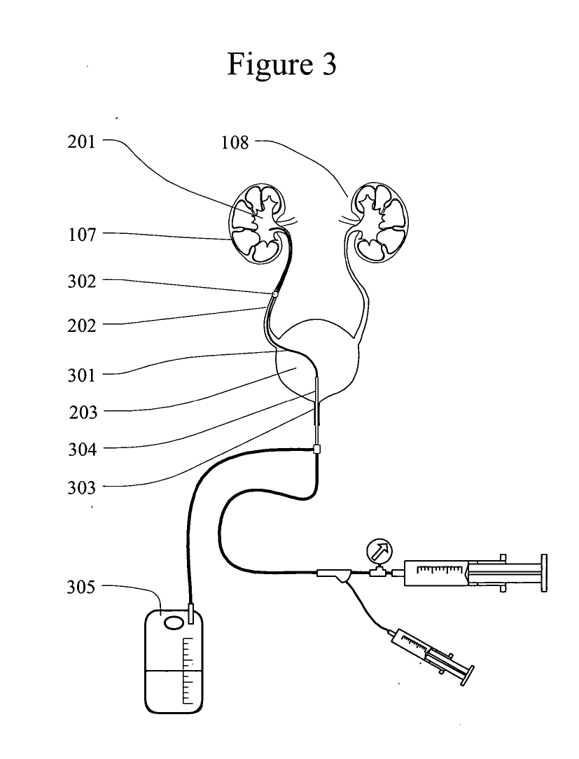

FIG. 3 illustrates an embodiment of a ureter catheter 301 that protects only one kidney by selectively elevating the pelvic pressure of one kidney. A ureteral catheter 301 is placed in a ureter 202 of the kidney 107. Placing a catheter in the ureter is somewhat more difficult than placing a catheter in the bladder via the urethra. It requires special surgical skills and instruments that are available to urologists. A laparoscopic procedure for the placement of a catheter in the ureter is described in U.S. Pat. No. 4,813,925, entitled Spiral Ureteral Stent. Catheter 301 is shown traversing into the bladder 203 through an introducer sheath 304 placed in the urethra 303. The catheter is further introduced into the ureter 202 with the tip of the catheter in the renal pelvis 201. Catheter 301 is equipped with an occluding balloon 302. The balloon 302 can be positioned in the ureter 202 (as shown) or in the pelvis 201 of the protected kidney. The balloon catheter system for the partial or complete ureteral occlusion is substantially the same as the design of other catheters uses by the invention. The unprotected kidney 108 continues to make urine that drains into the bladder. The sheath 304 is equipped with a drainage channel that allows urine to drain from the bladder 203 into the urine collection bag 305. It is understood that many other catheter design can be envisioned that will fulfill the same basic function of occluding and pressurizing the renal pelvis of a kidney. Elevated pressure in the bladder is therefore transmitted to both kidneys and causes the desired reduction of the GFR, increase of the RBF to GFR ratio and the relief of the renal medullary hypoxia in both kidneys.

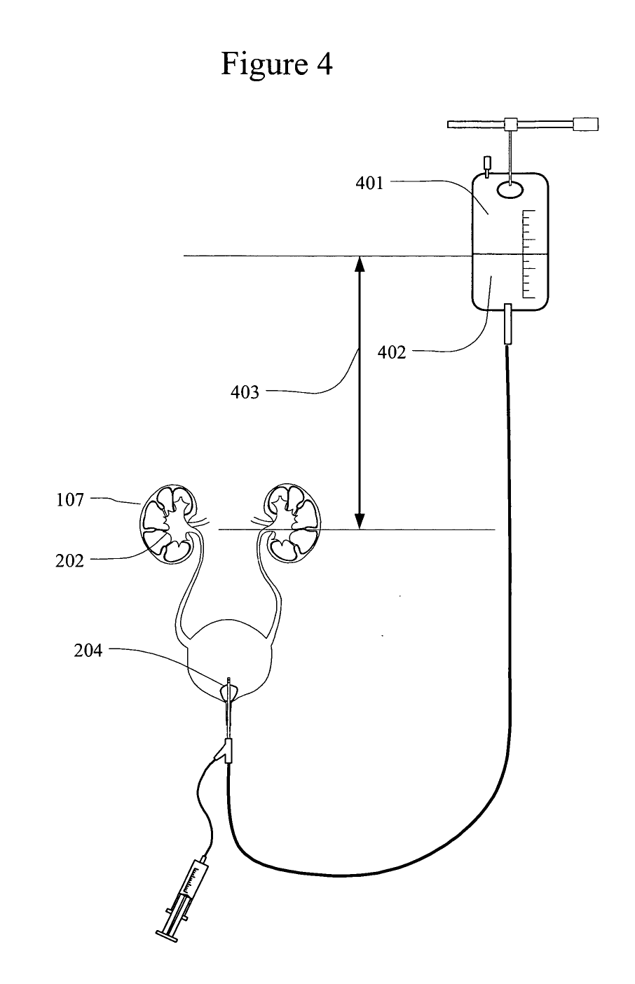

FIG. 4 illustrates a simple and inexpensive embodiment of a catheter inserted into the bladder that automatically maintains the pressure in the renal pelvis at a desired elevated level. The distal tip of the catheter 204 has an opening that allows the fluid communication between the renal pelvis 201 of the kidney 107 and the fluid bag 401. The bag 401 is filled with the hydraulic infusion solution 402, such as a sterile saline. The height difference 403 between the patient's kidney 107 and the level of fluid in the bag determines the hydraulic pressure in the renal pelvis. For example, if the hydraulic fluid has the specific gravity of water, the height difference 403 equal to 100 mm will generate the hydraulic pressure of 7.35 mmHg. It can be expected that for the efficient and safe protection of the kidney pelvic pressure in the range of 10 to 100 mmHg is desired. To achieve the desired increase in kidney pelvic pressure, the height of the bag 104 above the patient may be in a range of 13 centimeters (cm) to 140 cm. This method of controlling the pelvic pressure with an elevated fluid bag can be used with both bladder and individual ureter occlusion embodiments illustrated by FIGS. 2 and 3.

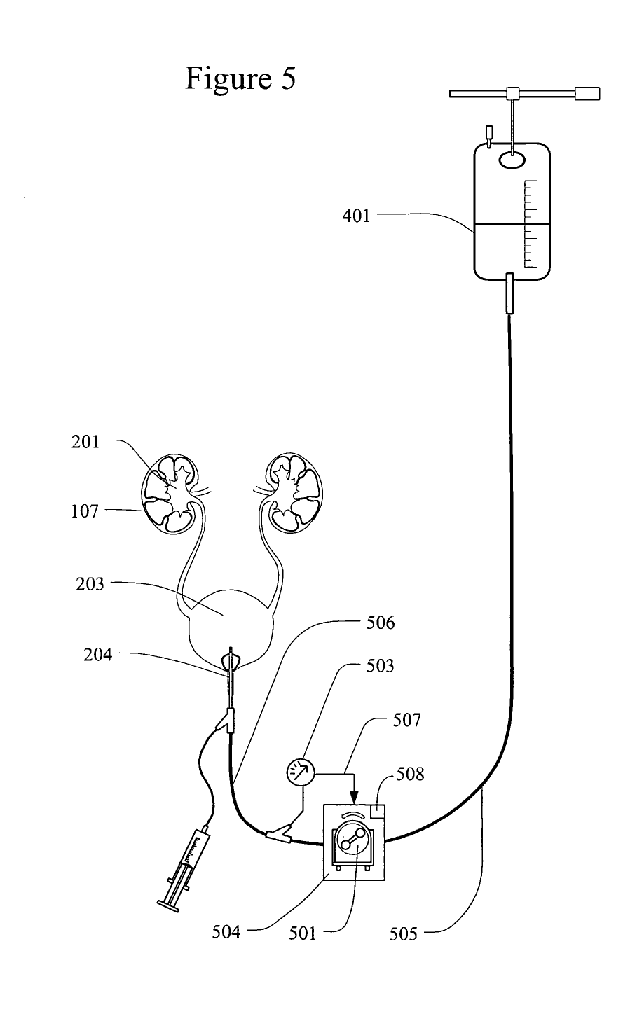

FIG. 5 shows a more complex embodiment. This embodiment may be preferred if more accurate control of the pelvic pressure over longer time is desired than may be available with the system shown in FIG. 4. The catheter 204 is connected to the fluid reservoir 401 via the fluid filled tubes 505 and 506. Fluid is infused into and drained from the bladder 203 by the electric motor controlled pump 501. The pump can be of any type commonly used to infuse IV medicine or to circulate blood. A suitable peristaltic roller pump is described, for example, in the U.S. Pat. No. 4,229,299. Pump rotation is controlled by a microprocessor based control system 508 inside the control console 504. The control console receives information from the pressure sensor 503 connected to the fluid tubing 506 and the catheter lumen extending to an outlet port in the bladder or urethra. Console controls the rotation of the pump based on the received pressure signal 507. The pressure signal may be indicative of a pressure in the bladder or urinary tract which affects the pressure in the catheter lumen. Sensor 502 can be a disposable blood pressure sensor (such as ones made by Merit Medical of Utah) that is used widely for invasive blood pressure measurement or similar to the compact tube-mounted pressure sensors described in U.S. Pat. Nos. 6,171,253 and 6,272,930.

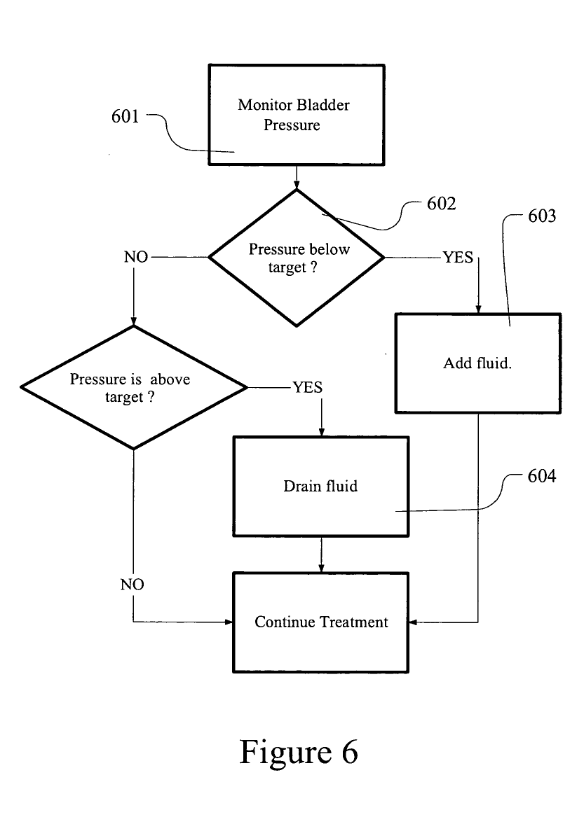

FIG. 6 illustrates a software algorithm embedded in a controller 508, e.g., a microprocessor, for the control console system 504 (FIG. 5). The controller and control console may be used in conjunction with any of the embodiments disclosed herein, including embodiments that include catheters having tips inserted in the renal artery, bladder and ureter. Fluid pressure is monitored 601 continuously using a pressure sensor 503, an amplifier and an analog-to-digital converter (Not shown). These are the standard components of a digital pressure monitor that need not be explained in detail. The processor is equipped with an internal clock. Information in digital form is supplied to the processor every 5-10 milliseconds. The software algorithm compares 602 the measured pressure to the target value set by the operator or calculated by the processor. The algorithm commands the inflation (infusion of fluid) 603 or deflation (draining of fluid) 604 of the bladder 203 based on the pressure feedback 601 with the objective of achieving the desired pressure target. Generally the algorithm achieves a pelvic pressure that is greater than 10 mmHg and less than 100 mmHg. Implementation of the algorithm illustrated by FIG. 6 can be easily achieved by applying methods known in the field of controls engineering. For example, classic process control algorithms such as the Proportional Integral (PI) controller can be used to maintain pressure at the target level. Control signals can be applied continuously or periodically to adjust the volume of fluid in the bladder. It can be expected that during the time of the procedure the bladder can stretch, contract, and leak fluid or that the patient's condition can change. In response to these changes the pelvic pressure target may change requiring a correction. It can be envisioned that the correction will be made automatically or by the operator based on the readings of pressure manometers but it is often preferred to have an automatic response to save time and increase safety.

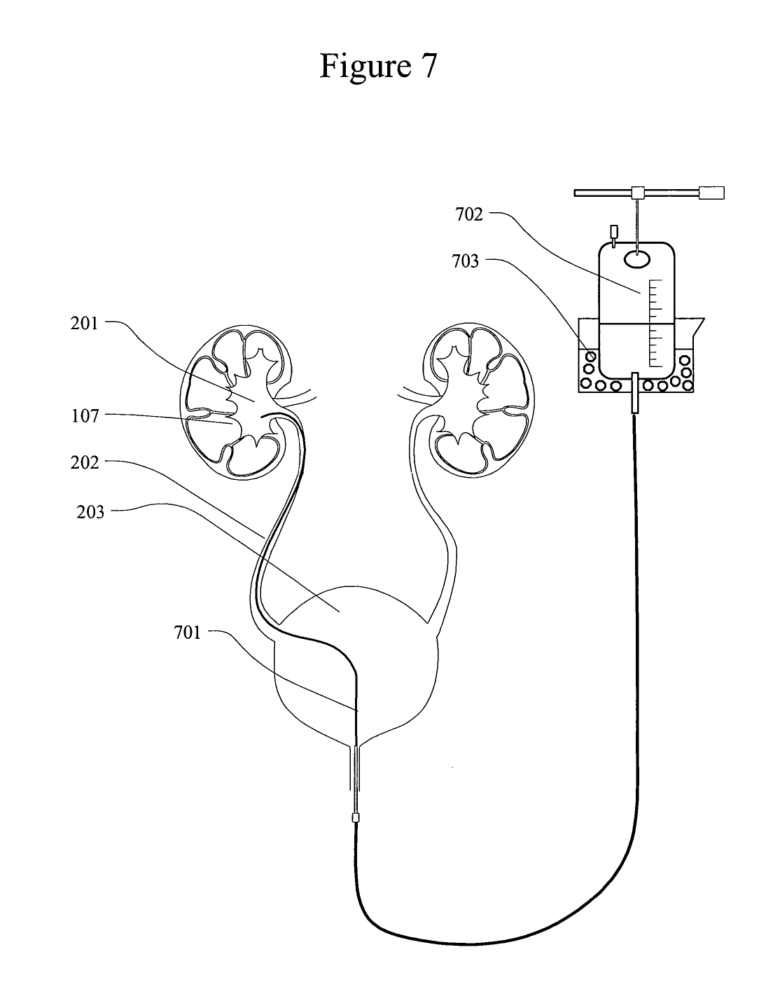

FIG. 7 illustrates the use of renal cooling as an adjunct to other disclosed embodiments or an independent method of protecting kidney from ischemia by reducing the metabolic energy consumption by the kidney. Protection of kidneys by cooling is well known. In surgery, when possible, kidneys are packed with ice to reduce the possibility of ARF. Experience with renal transplantation confirms that the kidney is well protected by cold and recovers from it well when it is re-warmed. The kidney 107 is cooled by continues infusion or irrigation with cold saline or other sterile solution into the renal pelvis 201. The ureteral catheter 701 has the distal tip residing in the pelvis. In the proposed embodiment the catheter 701 does not occlude the ureter 202 and the cooling solution infused into the renal pelvis is allowed to drain into the bladder 203. The cooling solution is stored in the bag 702 that is submerged into the ice water bath 703 to keep its temperature just above freezing.

The embodiments disclosed herein protect the kidney of a patient from an ischemic insult or treat the kidney by improving the ratio of the oxygen supply to demand as can be expressed for example by the ratio of renal blood flow to GFR. This goal is achieved by activating or provoking a physiologic response in the kidney normally associated with a disease state. As a result of this response, the GFR of the kidney (renal function) may be reduced in a lesser degree than the blood flow through the kidney. It may be that the renal blood flow to GFR ratio of one or two kidneys are artificially increased for the duration of the insult that can last from several hours to several weeks.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method to protect a kidney in a mammalian patient comprising:
a. artificially increasing pressure in a urinary tract of at least one kidney of the patient;
b. reducing a renal function of the kidney by maintaining the increased pressure, and
c. reducing the pressure in the urinary tract to increase the renal function above the reduced renal function.

2. A method as in claim 1 wherein the increase of pressure in the urinary tract is temporary.

3. A method as in claim 1 wherein the increase in the pressure in the urinary tract is reversible.

4. The method as in claim 1 wherein the urinary tract pressure is increased at least to a pressure of 10 to 20 cmH₂O above a pressure level in the urinary tract prior to the artificial increase in pressure.

5. The method as in claim 1 wherein the urinary tract pressure is increased prior to the administration of a contrast agent to the patient.

6. The method as in claim 5 wherein the urinary tract pressure is increased to protect the kidney from an insult.

7. The method as in claim 1 wherein the urinary tract pressure is increased prior to hypotensive surgery and the increased pressure is reduced after the surgery.

8. The method as in claim 1 wherein the urinary tract pressure is increased for at least one hour.

9. The method as in claim 1 wherein the urinary tract pressure is increased by artificially infusing fluid into a bladder of the patient.

10. The method as in claim 9 wherein infused fluid flows into the bladder of the patient without first flowing through the kidney.

11. The method as in claim 9 wherein the infused fluid flows into the bladder through a urethra of the patient prior to entering the bladder.

12. The method as in claim 9 further comprising maintaining an increased pressure in the bladder by applying an elevated pressure to the infused fluid in the bladder.

13. The method as in claim 12 wherein the elevated pressure of the infused fluid is applied by gravity.

14. The method as in claim 12 wherein the infused fluid flows from a container elevated above the patient and flows from the container into the bladder.

15. The method as in claim 14 wherein the container is elevated about the patient a distance in a range of range of 13 centimeters(cm) to 140 cm above the patient.

16. The method as in claim 14 wherein the infused fluid flows from the container into the bladder due to gravity.

17. The method as in claim 1 wherein increasing the urinary tract pressure further comprises artificially distending the bladder of the patient.

18. The method as in claim 17 wherein artificially distending the bladder further comprises artificially infusing fluid into the bladder.

19. The method as in claim 1 wherein increasing the urinary tract pressure further comprises at least partially obstructing a flow of urine from the kidney and through the urinary tract.

20. The method as in claim 1 wherein increasing the urinary tract pressure further comprises at least partially obstructing a flow of urine from the bladder.

21. A method to prevent or treat contrast nephropathy in a mammalian patient undergoing a radiographic procedure comprising:
a. artificially increasing pressure in a urinary tract of at least one kidney of the patient;
b. injecting the contrast agent into a blood vessel of the patient, and
c. reducing pressure in the urinary tract of the kidney.

22. A method as in claim 21 further comprising reducing a renal function of the during a period in which the contrast agent is in the blood of the patient.

23. A method as in claim 21 further comprising, prior to step (a), identifying the patient from a group of patients suffering from one or more of a group of illnesses consisting of chronic renal disease, diabetes and old age, wherein the identified patient is determined to be a particularly risk during injection of a contrast agent.

24. A method as in claim 21 wherein reducing the pressure returns the urinary tract to a pressure that existed before injection of the contrast agent.

25. A method as in claim 21 wherein the increase of pressure in the urinary tract is temporary.

26. A method as in claim 21 wherein the increase in the pressure in the urinary tract is reversible.

27. A method as in claim 21 wherein steps (a), (b) and (c) are preformed sequentially.

28. The method as in claim 21 wherein the urinary tract pressure is increased at least to a pressure of 10 to 20 cmH₂O above a pressure level in the urinary tract before step (a).

29. The method as in claim 21 wherein the urinary tract pressure is increased prior to the administration of the contrast agent to the patient.

30. The method as in claim 29 wherein the urinary tract pressure is a pressure in a bladder of the patient.

31. The method as in claim 21 wherein the urinary tract pressure is increased for at least one hour.

32. The method as in claim 21 wherein the urinary tract pressure is increased by artificially infusing fluid into a bladder of the patient.

33. The method as in claim 32 wherein the infused fluid flows into the bladder of the patient without first flowing through the kidney.

34. The method as in claim 32 wherein the infused fluid flows into the bladder through a urethra of the patient prior to entering the bladder.

35. The method as in claim 33 further comprising maintaining an increased pressure in the bladder by applying an elevated pressure to the infused fluid in the bladder.

36. The method as in claim 35 wherein the elevated pressure of the infused fluid is applied by gravity.

37. The method as in claim 36 wherein the infused fluid flows from a container elevated above the patient and flows from the container into the bladder.

38. The method as in claim 37 wherein the container is elevated about the patient a distance in a range of range of 13 centimeters(cm) to 140 cm above the patient.

39. The method as in claim 37 wherein the infused fluid flows from the container into the bladder due to gravity.

40. The method as in claim 37 further comprising regulating a flow of the infused fluid into the bladder by an adjustable pump.

41. The method as in claim 35 wherein increasing the urinary tract pressure further comprises artificially distending the bladder of the patient.

42. The method as in claim 41 wherein artificially distending the bladder further comprises artificially infusing fluid into the bladder.

43. The method as in claim 35 wherein increasing the urinary tract pressure further comprises at least partially obstructing a flow of urine from the kidney and through the urinary tract.

44. The method as in claim 35 wherein increasing the urinary tract pressure further comprises at least partially obstructing a flow of urine from the bladder.

45. A method to inhibit a natural function of a kidney of a patient during surgery:
a. artificially increasing a pressure in a urinary tract of at least one kidney of the patient,
b. performing the surgery on the patient, and
c. reducing pressure in the urinary tract of the kidney to substantially a pressure level existing before step (a).

46. A method as in claim 45 wherein the increase of pressure in the urinary tract is temporary.

47. A method as in claim 45 wherein the increase in the pressure in the urinary tract is reversible.

48. The method as in claim 45 wherein the urinary tract pressure is increased at least to a pressure of 10 to 20 cmH₂O above a pressure level in the urinary tract prior to step (a).

49. The method as in claim 45 wherein the urinary tract pressure is a pressure in a bladder of the patient.

50. The method as in claim 45 wherein the urinary tract pressure is increased for at least one hour.

51. The method as in claim 45 wherein the urinary tract pressure is increased by artificially infusing fluid into a bladder of the patient.

52. The method as in claim 51 wherein the infused fluid flows into the bladder through a urethra of the patient prior to entering the bladder.

53. The method as in claim 51 further comprising maintaining an increased pressure in the bladder by applying an elevated pressure to the infused fluid in the bladder.

54. The method as in claim 53 wherein the elevated pressure of the infused fluid is applied by gravity.

55. The method as in claim 54 wherein the infused fluid flows from a container elevated above the patient and flows from the container into the bladder.

56. The method as in claim 55 wherein the container is elevated about the patient a distance in a range of range of 13 centimeters(cm) to 140 cm above the patient.

57. The method as in claim 51 further comprising regulating a flow of the infused fluid into the bladder by an adjustable pump.

58. The method as in claim 45 wherein increasing the urinary tract pressure further comprises artificially distending the bladder of the patient.

59. The method as in claim 58 wherein artificially distending the bladder further comprises artificially infusing fluid into the bladder.

60. The method as in claim 45 wherein increasing the urinary tract pressure further comprises at least partially obstructing a flow of urine from the kidney and through the urinary tract.

61. The method as in claim 45 wherein increasing the urinary tract pressure further comprises at least partially obstructing a flow of urine from the bladder.

62. The method as in claim 45 wherein increasing the urinary tract pressure further comprises increasing the pressure in the urinary tract in a range of 15 cmH₂O to 150 cmH₂O.

63. The method as in claim 45 wherein increasing the urinary tract pressure further comprises increasing the pressure in the urinary tract for at least 30 min but less than 24 hours before the step of restoring pressure.

64. The method as in claim 45 wherein steps (a), (b) and (c) are preformed in sequence.

65. The method as in claim 45 wherein the surgery begins prior to increasing the pressure in the urinary tract.

66. The method as in claim 45 wherein the surgery is substantially completed before reducing the pressure in the urinary tract.

67. A system for preventing or treating acute renal failure in a mammalian patient comprising:
means for artificially increasing pressure in the urinary tract of at least one kidney to reduce a renal function of the kidney;
monitoring means for sensing and displaying a pressure related to the pressure in the means for artificially increasing pressure, and
means for restoring said pressure and to restore the renal function.

68. A system as in claim 67 further comprising mean for maintaining said increased pressure at a predetermined pressure.

69. A system as in claim 67 wherein said means for maintain said increased pressure further comprises means for adjusting the predetermined pressure.

70. A system as in claim 67 wherein the means for artificially increasing pressure further comprises means for artificially increasing pressure means for increasing a bladder pressure.

71. A system as in claim 67 wherein the means for artificially increasing pressure further comprises means for artificially infusing a fluid in to a bladder of the patient.

72. A system as in claim 71 wherein the means for artificially increasing pressure further comprises a catheter having an expandable device at a distal section of the catheter.

73. A system as in claim 72 wherein the expandable device is insertable in a bladder of the patient.

74. A system to treat at least one kidney of a mammalian patient, said system comprising:
a catheter positionable in a urinary tract leading from the at least one kidney;
said catheter having a distal tip with an occlusion device and a pressure sensing port, wherein the occlusion device has an occlusion mode and a passive mode;
a pressure sensor in fluid communication with the pressure sensing port, and
said occlusion device operating in said occlusion mode to elevate a pressure in the urinary tract to a pressure sufficient to inhibit a renal function.

75. A system as in claim 74 wherein the occlusion device is a balloon and further comprising:
a balloon fluid injector in fluid communication with the balloon, and
an actuator for controlling an injection of the balloon fluid into the balloon to switch the occlusion device to the occlusion mode.

76. A system as in claim 74 further comprising a controller switching the occlusion device between the occlusion mode and the passive mode.

77. The system as in claim 74 wherein the occlusion device when in the occlusion mode at least partially obstructs urine output.

78. The system as in claim 77 wherein the occlusion device when in the occlusion mode at least partially obstructs urine output from a bladder of the patient.

79. The system as in claim 74 further comprising a radiocontrast injector and wherein the occlusion device is operated in the occlusion mode during radiocontrast injection.

80. The system as in claim 74 wherein the occlusion device is operated in the occlusion mode during a surgical procedure.

81. A system to temporarily reduce a natural function of at least one kidney of a mammalian patient, said system comprising:
a catheter positionable in a bladder of the patient;
said catheter having a distal end with an occlusion device, and an infusion fluid port;
a pressure device coupled to a fusion fluid supply and elevating the pressure of the infused fluid feed to the fluid port,
wherein the occlusion device has an occlusion mode and a passive mode;
an infusion fluid supply connectable to a proximal section of the catheter and in fluid communication with the fluid port, wherein
the occlusion device has an occlusion mode to occlude the bladder while the infusion fluid is infused into the bladder, and a release mode for allowing the bladder to drain of the infused fluid.

82. A system as in claim 81 further comprising a pressure sensor in fluid communication with the infusion fluid port, wherein said sensor generates a signal indicative of a pressure in the bladder.

83. A system as in claim 81 wherein the occlusion device is a balloon.

84. A system as in claim 81 wherein the pressure device is a pump coupled to a fluid tube extending from the fluid supply to the catheter.

85. A system is in claim 81 wherein the pressure device is a support for the container, wherein the support is elevated above the patient.

86. A system as in claim 81 wherein the infusion fluid supply further comprises:
a container for the infusion fluid coupled to a proximal section of the catheter and in fluid communication with the fluid port;
a conduit extending from the container to the proximal section of the catheter, and
an elevated fluid container support, wherein the container is supported above the patient.

87. A system as in claim 86 wherein the container is supported a predetermined distance is a range of 13 centimeters(cm) to 140 cm above the patient.

88. A system as in claim 86 wherein the container is gravity fed to the catheter.

89. A system as in claim 86 further comprising a pump operatively coupled to for moving fluid in the container to the catheter.

90. A system as in claim 86 further comprising a pump for moving fluid in the container to the catheter.

91. A method to elevate pressure in a urinary at least one kidney of a mammalian patient, said method comprising:
a. inserting a catheter tip into a ureter of the patient;
b. obstructing fluid flow from the kidney and through the ureter by with the tip;
c. elevating a fluid pressure in the ureter by the obstructed fluid flow;
d. affecting a function of the kidney by the elevated fluid pressure, and
e. releasing the obstructed fluid through the ureter by deactivating or removing the catheter tip, and
f. resuming the kidney function after releasing the obstructed fluid.

92. A method as in claim 91 wherein the elevated fluid pressure further comprises injecting fluid through the catheter and into the ureter to elevate the fluid pressure in the ureter and at an outlet of the kidney, and said method further comprises draining the fluid injected into the ureter through the kidney while the fluid continues to be injected into the ureter.

93. A method as in claim 92 wherein the catheter tip is further connected to a pressure sensor detecting a fluid pressure in the ureter and said method further comprises:
monitoring the fluid pressure the ureter while the occlusion device is activated;
injecting the fluid into the ureter if the fluid pressure in the ureter is below a predetermined lower pressure threshold, and
draining fluid from the ureter if the pressure in the bladder is below a predetermined higher pressure threshold.

94. A method as in claim 91 wherein steps (b), (c) and (d) coincide with a radiocontrast injection into the patient.

95. A method as in claim 92 wherein the fluid is injected into the ureter by gravity and from a fluid bag elevated above the patient.

96. A method as in claim 95 wherein the fluid bag is elevated above the patient in a range of 130 cm to 140 cm.

97. A method as in claim 91 further comprising:
cooling the fluid to be injected into the ureter and
cooling the kidneys with the cooled fluid injected into the ureter.
